(19) United States
(12) Patent Application Publication
(10) Pub. No.: US 2005/0106268 A1
(43) Pub. Date: May 19, 2005

(54) Mold and microbial remediation using dry ice blasting

(76) Inventor: Jay T. Armstrong, Wichita, KS (US)

Correspondence Address: Patrick D. Kelly, 11939 Manchester # 403, St. Louis, MD, 63131, US

(21) Appl. No.: 10/799,545

(22) Filed: Mar. 11, 2004

Related U.S. Application Data
(60) Provisional application No. 60/453,781, filed on Mar. 11, 2003.

(51) Int. Cl. A61K 33/00; B24C 9/00

(52) U.S. Cl. 424/700; 451/88

(57) ABSTRACT

A method for cleaning noxious colonies of mold and other microbes out of buildings is disclosed, using “dry ice blasting”. This method involves pumping a gas stream containing small particles of dry ice (solidified carbon dioxide, or CO₂) through a high-speed nozzle that can be directed at any surface that needs to be cleaned. This type of equipment previously has been used for cleaning charred surfaces and coating layers of soot and smoke off of wooden and other surfaces, in a building that has suffered a fire, but it has not previusly been used for mold remediation. It is highly effective for cleaning uneven surfaces, such as the surfaces of unfinished lumber, using a controllable a combination of pressure, velocity, and flow rates that can effectively soften, abrade, and blow away any surface layers that do not have the hardness of normal wood.

### RELATED APPLICATION

This application claims the benefit under 35 USC 119(e) of provisional application 60/453,781, filed on Mar. 11, 2003.

### FIELD OF THE INVENTION

This invention involves machinery for cleaning building interiors and other structures that cannot be moved, to remove colonies of mold and other unwanted microbes. This is done by using machinery that pumps dry ice particles through a hose and emits them through a nozzle at high speed, in a manner comparable to sand blasting.

### BACKGROUND OF THE INVENTION

Growths of mold and other noxious microbes inside the walls of buildings have become major problems in America and elsewhere. These types of growths generate noxious odors and can trigger serious health problems (including asthma attacks and other respiratory problems) among people who live in, work in, or merely visit such buildings. In addition, over a span of years, a combination of microbes and moisture can inflict serious structural damage on wood, dry wall, and other structural components.

Accordingly, when a home or other building is discovered to contain a major colony of mold or other microbes, the infestation must be removed, even if large costs must be incurred, to keep the problem from continuing to grow until it renders the building uninhabitable.

For convenience, the discussion below will focus on mold colonies, since those pose the worst and most common threats to buildings. However, it should be understood that this invention can also be used to clean and remove other types of microbial growths and infestations as well, including bacterial infestations, fungal infestations, etc.

The terms infestation, growth, and colony are used interchangeably herein, to refer to any type of mold or other microbial colony that has reached a size that requires a specialized operation to remove the colony and clean the spot where it had been growing, to prevent, retard, or minimize its risk of recurrence.

If a large colony of mold or other microbes begins to grow in an area that can be readily seen, the people who live or work in the building will simply clean out that colony. Therefore, mold and other microbial infestations in buildings pose the most serious problems when they occur in areas that are not visible to occupants during normal habitation, work, or other use of the building. Such non-visible areas include, but are not limited to, the gaps and empty spaces that exist between the studs, beams, joists, casings, and other supports that help create and support a typical wall, floor, or ceiling, in a home or other building. Those gaps and empty spaces typically are covered up and hidden by dry-wall, sheet-rock, particle board, plywood, paneling, suspended ceiling panels, or other comparable materials.

Microbes are everywhere, and it is impossible to prevent them from being present in any location that cannot be completely sterilized. The major limitation on whether the microbes will grow to a point of forming noxious colonies, in some particular hidden space, usually hinges on how much water (usually in the form of air-borne humidity) is available to the microbes in that hidden space. If a hidden space remains dry, it usually will not suffer from substantial growth of mold or other colonies. However, sources of water can become available to the microbes that are present in a hidden space, due to any of various types of problems (such as a leak in a roof, pipe, or dryer vent, condensation that occurs when humid air contacts a cold water pipe, air conditioner duct, or outer wall, or some problem that involves groundwater, stormwater runoff, a cracked drainpipe, etc.). If that type of water supply becomes available to the microbes in a hidden space, the water supply can enable the growth of a large and noxious colony of mold or other microbes.

If a large mold colony is discovered inside a building wall, the removal operation typically is carried out in five major stages. First, one or more panels, sheets, or other pieces of relatively inexpensive materials that hide, shelter, and protect the colony (such materials include paneling, dry wall, etc., and are referred to herein as “covering components”) usually must be removed, to expose the underlying studs, joists, rafters, casings, or other structural supports (referred to herein as “supporting members”) in a way that will expose the microbial colony, as much as necessary or practical. The old covering components, which usually are relatively inexpensive and replaceable without requiring major structural repairs, typically are discarded.

Next, one or more mechanical means (such as scraping, brushing, wet/dry vacuuming, etc.) are normally used, to remove the bulk of the microbial mass. In the third stage, the partially cleaned surfaces are washed with a microbicidal detergent, to kill as many of the remaining microbes as possible. Fourth, unless the microbicidal detergent has already created a fairly heavy layer of a antimicrobial residue on any wood or other surfaces, those surfaces are sprayed or coated with a microbicidal coating that will leave a residue, which preferably should remain in place for months or even years, to prevent or minimize future infestations. In the fifth and final stage, the dry wall, paneling, or other surfaces are replaced, to return the building to a normal appearance and habitable condition.

In most cases, this type of remedial operation becomes a major and expensive undertaking. Because of the health risks that are involved (which include the risk of people breathing in microbes, spores, and mycotoxins, all of which usually become airborne in large quantities when a large microbial colony is disturbed), and because of the risk of spreading the noxious microbes to additional sites in the building unless careful steps are taken to prevent them from spreading and forming still more colonies, this type of remediation work can be done safely and effectively only by trained professionals, who usually need to wear respirators and other protective gear while they work. These workers typically seal off a room before they begin working on it, using adhesive tape and sheets of plastic, and they normally must use special types of fans and filters, to suck out any air and pass it through filters that will trap any airborne microbes, so they can be destroyed rather than releasing them into the outside air. Because of these types of precautions and procedures, this type of remediation usually costs multiple thousands of dollars, and it usually requires a family to vacate its home for at least several days.

In addition, when a room or building is discovered to be infested by a mold or other noxious microbial colony, a professional remediator usually will carry out a fairly thorough inspection of any and all hidden areas that have elevated risks of being subjected to water leakage, high moisture content, or other contributing factors that can support an infestation. In many situations, these types of inspections can become complex and extensive, and often involve fiber-optic “snake”-type devices, for visual inspections of hidden areas, as well as various types of humidity and moisture probes.

None of these operations are perfect, in their efficiency. Due to various factors, it is impossible for any mold removal or remediation program to achieve a complete and total 100% kill of all of the cells and spores that might be able to start a new colony, if they survive. The factors that work together to limit and hinder the efficacy and reliability of any remediation operation include: (i) the rough and uneven surfaces of unfinished pine, particle board, and other moderately soft materials that are widely used in building construction; (ii) the ability of even a single isolated microbe (which is far too small for anyone to see) to reproduce into a huge new colony; (iii) the tendency of mold and other microbes to grow in relatively thick layers that, during a removal operation, will protect cells and spores that are located beneath the surface; and (iv) the limitations on how toxic and lethal a practical microbicidal chemical can be, if it must be used with reasonable safely in a household environment where humans and pets might be unintentionally exposed.

It should also be noted that it is extremely difficult to kill microbial spores, during a building remediation. Spores are small, dehydrated versions of microbial cells. They are comparable to the small, hard seeds found in apples, oranges, watermelons, and other fruits, melons, and berries. Spores are common among opportunistic microbes, because they can enable a microbial colony (or its descendants) to survive for many years (or even decades, or in some cases centuries) in a completely dormant state, if sufficient moisture or nutrients are not present. Then, years later, the spores can suddenly revive, switch back into an active state, and begin growing and reproducing, as soon as moisture and/or nutrients become available again.

Because spores are dormant, they do not actively take anything into their cells, and they usually are enclosed within a relatively tough and durable dehydrated coating layer. Because of how those factors work together, the coating layers of spores generally will not allow any molecules larger than water to penetrate through the coating, and into the interior of the cell, where microbial proteins and enzymes might offer targets for microbicidal poisons. Since any type of microbicidal toxin will necessarily be much larger than a water molecule, this prevents most types of microbicides from being able to reliably kill spores. The only reliable ways to kill spores are through sustained boiling or autoclaving, or by the use of powerful bleaches or detergents that will disrupt the coating layers of the spores.

Clearly, boiling or autoclaving of the struts and walls of a building are not possible. Bleaches and detergents are indeed used during building remediations, but they have two drawbacks. First, they tend to leave behind unpleasant odors; and second, since they are surface-acting agents, they need to be able to penetrate all the way through a microbial colony, and reliably reach cells even in layers and tiny crevices that are deeply buried, hidden, and protected.

For all of these reasons, there is a pressing need for better ways to achieve improved cleaning of wood and similar porous or uneven surfaces, in buildings that have hidden spaces that have become infested by large colonies of mold or other noxious microbes.

One object of this invention is to disclose that dry ice blasting offers a practical and highly effective method for removing large colonies of mold or other noxious microbes from buildings.

Another object of this invention is to provide a method for improved cleaning of noxious colonies of mold and other microbes, from hidden spaces inside the walls, floors, or other structures of buildings.

A third object of this invention is to disclose an improved method for cleaning and removing noxious colonies of mold and other microbes from hidden spaces in buildings, in order to enable better chemical decontamination of the surfaces where the colonies previously grew.

These and other objects of the invention will become more apparent through the following summary and description.

### SUMMARY OF THE INVENTION

A method for cleaning noxious colonies of mold and other microbes out of buildings (including homes) is disclosed, using dry ice blasting. This method involves pumping small hardened particles of dry ice (solidified carbon dioxide, or CO₂), roughly the size of rice grains, through a hose with a nozzle that can be directed at any surface that needs to be cleaned. This type of equipment previously has been used for cleaning charred surfaces and coating layers of soot and smoke off of wooden and other surfaces, in a building that has suffered a fire. It uses a combination of pressure, velocity, and flow rate that will effectively soften, abrade, and blow away any surface layers that do not have the hardness of normal wood. In addition, because of the size ranges and high velocities of the dry ice particles, they are very effective in penetrating into and cleaning uneven surfaces (such as the surfaces of unfinished lumber) and any cracks, crevices, gaps, and other spaces where hidden microbes would be difficult to reach and remove. In most cases, this type of dry ice blasting operation requires a room to be sealed off, using adhesive tape and plastic sheeting, and provided with an exhaust system that will create a mild vacuum, which will continuously suction the atmosphere out of the room, and into a filter system that will trap any microbes that became airborne during the dry ice blasting operation. This type of operation will require the workers to wear breathing equipment, if they are in the room during a blasting operation.

### DETAILED DESCRIPTION

As summarized above, this invention discloses that dry ice blasting provides a highly effective method for cleaning and eliminating noxious colonies of mold and other microbes, from homes and other buildings. This method involves pumping small hardened particles of dry ice (solidified carbon dioxide, or CO₂), roughly the size of rice grains, through a hose with a nozzle that can be directed at any surface that needs to be cleaned.

All of the necessary equipment and methods have previously been developed, for uses that are entirely different from the new use disclosed herein. In particular, an entirely suitable dry ice blasting system is already commercially available, from the Cryokinetics division of The L.A.W. Group, Inc., in Wichita, Kans. (www.cryokinetics.com). Suitable models that are sold by Cryokinetics include the Delta V-1.

This type of system was initially developed for cleaning soot and smoke off of wooden and similar surfaces in a building that has suffered a fire. In addition to removing layers of soot and smoke from such surfaces, dry ice blasting can also remove the weakened surface layers of charred wood, without significantly damaging or weakening any underlying layers of wood that remain solid and strong. Therefore, dry ice blasting has become a preferred method of cleaning out the interiors of buildings that have suffered fires.

Accordingly, the procedures and equipment that were developed for those types of cleaning operations, after a fire, can be readily adapted to microbial remediation operations.

Before a dry ice blasting operation can commence, sheets of plastic are emplaced across any windows, doors, or other room openings, using strong and wide adhesive tape, to seal off the room except for a single point of egress (entry and exit). A large fan is placed at the egress location, in a manner that will allow it to suction carbon dioxide and airborne microbes out of the room. This fan preferably should draw air through a “HEPA” exhaust system (this acronym stands for High Efficiency Particulate Air filter). That filter will trap any microbes and other particulates that have become airborne during the blasting operation, and it will be positioned in the room and/or provided with an outlet duct so that the carbon dioxide, air, and other gases that pass through the filter will be emitted to the outside atmosphere. The fan should be powerful enough to create a vacuum in the room which will generally be in the range of about 20 inches of water (“inches of water” refers to the vertical gap that will occur in a U-shaped tube filled with water, if one end of the U-shaped tube is open to the outside atmosphere, while the other end of the tube is subjected to the vacuum). This will cause the atmosphere in the room to be continuously suctioned, and passed through the HEPA filter.

After the plastic sheeting enclosure (often referred to as a “tent”) and the fan-and-filter system are in place and ready, the hidden areas where the microbes are actively growing should be exposed, to the greatest extent practicable. In most building remediations, this typically will require paneling, dry-wall, sheet-rock, ceiling panels, and other similar materials to be removed from any walls, floors, or ceiling areas that are infested, or that enclose undesirably high moisture levels. This removal operation will expose any studs, beams, joists, casings, and other internal structures and supports, as well as any electrical wiring, plumbing, heating or air conditioning ducts, and other building components that normally are hidden by the paneling, dry wall, etc. After this operation has been completed, the room is usually suctioned out for a period of time (such as overnight), to remove any dust that was generated during that preparatory work.

When the blasting operation is ready to begin, an air compressor that can generate at least about 100 pounds per square inch of pressure is positioned outside the room that will be cleaned. These types of air compressors are commercially available; typically, they use diesel or gasoline engines, and are mounted on a wheeled dolly or hand-truck that is small enough to be lifted to any floor in an office building, by a standard elevator. When a home is being remediated, this compressor is usually positioned outside the building, so that the engine exhaust will remain outside the building.

A pneumatic hose (which can be as long as desired) is coupled at one end to the air compressor, and at the other end to the dry ice blasting unit. The dry ice blasting unit usually should be placed in the room being cleaned, or relatively close to the room, so that the dry ice particles will not have to travel long distances in a hose before they are emitted from the blasting nozzle.

When the dry ice blasting machine is turned on, the only control on the machine that the operator will need to adjust, in most situations, will be the flow rate (also called speed rate) of the dry ice. This can be expressed in pounds per minute, and a typical flow rate that works well for most types of cleaning uses is in the range of about 3 lb/min.

After a proper flow rate has been established, the operator uses heavy insulated gloves to hold and point the nozzle, by means of a moderately long tube, pole, or other handle-type device, which usually will also be insulated. The two main parameters that the operator will control, while actually cleaning the surfaces that need to be cleaned, will be (1) the distance of the nozzle tip from the surface being cleaned, and (2) the speed at which the operator moves the nozzle, over or across the surface being cleaned. The type of motion that is required and preferred is directly comparable to using a high-pressure water sprayer, to power-wash a deck or other outdoor surface. Proper use of a high-pressure nozzle of this type is not difficult to learn, and anyone who does it for several minutes will soon realize that two offsetting factors need to be balanced against each other. Better and more thorough cleaning can be achieved by holding the nozzle stationary, and close to the surface being cleaned; however, if the nozzle is held too close to a single spot for too long, then the water jet (during outdoor power-washing) or dry ice blasting (during smoke or mold remediation, inside a room) will begin to erode and damage the wood or other material that is being cleaned. By using continuing visual inspection of the area that is being cleaned, a trained operator can quickly learn to develop and sustain a reasonable combination of motion and distance that can achieve the desired results without damaging the underlying wood or other surfaces.

During a dry ice blasting process, gaseous carbon dioxide will be generated in fairly large quantities, when the dry ice particles sublimate and vaporize (this happens immediately, when the particles impact against a solid surface). This gaseous CO₂ will carry airborne microbes that were dislodged by the blasting operation. When it is drawn through the HEPA filter by the exhaust fan in the room, the microbes will be collected on the filter, and the gaseous CO₂ will pass through the filter and be vented to the outside atmosphere.

This type of operation usually requires workers to wear breathing equipment while in a room that is being cleaned by a blasting operation, to prevent the inhalation of microbes. Typically, a full-face respirator is used, and it should be equipped with an organic and HEPA cartridge; these are available commercially, from companies such as MSA (www.msanet.com). Workers should also have an oxygen analyzer available, to ensure that the oxygen supply in a room does not fall below safe levels when displaced by carbon dioxide; however, since the seals that are typically made by plastic sheeting and adhesive tape in a normal building are not truly air-tight, oxygen depletion is usually not a substantial danger.

Those skilled in the art are either already familiar with, or can quickly learn, the preferred procedures for carrying out this type of microbial remediation. All of the procedures, equipment, and supplies that are necessary or preferred for carrying out this type of microbial remediation are already known, and can be readily adapted from similar procedures that use dry ice blasting for other purposes, such as cleaning up fire and smoke damage, after a fire in a building.

However, it should be noted and understood that, to the best of the Inventor's knowledge and belief: (i) dry ice blasting has not previously been used, to remove colonies of mold and other noxious microbes from inside buildings; and, (ii) dry ice blasting appears to provide a nearly ideal means for microbial remediation inside homes or other buildings, and can clean a non-polished surface (such as wood) more thoroughly and effectively than any other known method of treatment. It leaves behind no residue, and when the blasting operation has been completed, the cleaned surfaces can be sprayed or otherwise treated, if desired, with any suitable type of disinfectant or other antimicrobial compound, to prevent or retard the subsequent growth of any new colonies.

Thus, there has been shown and described a new and useful means for using dry ice blasting to remove colonies of mold or other noxious microbes from inside buildings or other structures. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A method for cleaning noxious colonies of mold and other microbes out of buildings, comprising the use of dry ice blasting to dislodge at least one microbial growth from at least one solid surface supporting such growth.

2. The method of claim 1, wherein said dry ice blasting to dislodge at least one microbial growth is carried out after at least one covering component has been removed, to expose microbial growth on at least one surface of a supporting member.

3. The method of claim 1, wherein said dry ice blasting is carried out within a room after an exhaust fan has been provided and positioned in the room in a manner that will suction carbon dioxide and airborne particles out of the room and that will pass the carbon dioxide and airborne particles through a particulate filter.

4. A method for cleaning noxious colonies of mold and other microbes out of buildings, comprising the use of particulate carbon dioxide to dislodge at least one microbial growth from at least one solid surface supporting such growth.

5. The method of claim 1, wherein said particulate carbon dioxide is passed through a movable nozzle at high velocity, in a manner that dislodges microbes from supporting surfaces.

6. The method of claim 4, wherein said particulate carbon dioxide is passed through the movable nozzle after at least one covering component has been removed, to expose microbial growth on at least one surface of a supporting member.

7. The method of claim 4, wherein said said particulate carbon dioxide is passed through the movable nozzle after an exhaust fan has been provided and positioned in the room in a manner that will suction carbon dioxide and airborne particles out of the room and that will pass the carbon dioxide and airborne particles-through a particulate filter.
